(19) United States
(12) Patent Application Publication
(10) Pub. No.: US 2016/0129030 A1
(43) Pub. Date: May 12, 2016

(54) TREATMENT OF MTOR HYPERACTIVE RELATED DISEASES AND DISORDERS

(71) Applicant: THE BRIGHAM AND WOMEN'S HOSPITAL, INC., Boston, MA (US)

(72) Inventors: Elizabeth P. HENSKE, Cambridge, MA (US); Douglas A. MEDVETZ, Brookline, MA (US)

(73) Assignee: THE BRIGHAM AND WOMEN'S HOSPITAL, INC., Boston, MA (US)

(21) Appl. No.: 14/898,357

(22) PCT Filed: Jun. 11, 2014
(86) PCT No.: PCT/US14/41902

§ 371 Date: Dec. 14, 2015

Related U.S. Application Data
(60) Provisional application No. 61/835,023, filed on Jun. 14, 2013.
(60) Provisional application No. 61/939,906, filed on Feb. 14, 2014.

(51) Int. Cl. A61K 31/7034; A61K 31/40; A61K 31/404; A61K 31/167; A61K 31/192; A61N 5/10; A61K 31/357; A61K 31/135; A61K 31/472; A61K 45/06; G01N 33/573; A61K 31/198; A61K 31/5375

(52) U.S. Cl. CPC A61K 31/7034; A61K 31/198; A61K 31/40; A61K 31/404; A61K 31/167; A61K 31/192; A61K 31/5375; A61K 31/357; A61K 31/135; A61K 31/472; A61K 45/06; G01N 33/573; A61N 5/10; G01N 2333/912; G01N 2800/7028; G01N 2800/52

(57) ABSTRACT

Embodiments disclosed herein provide compositions and methods for treating cancer having deregulated mTOR signaling or mTOR hyperactivity, e.g., lymphangioleiomyomatosis (LAM), LAM/TSC or treating and/or management of tuberous sclerosis complex (TSC). Such methods and compositions comprise at least one compound selected from the group consisting of nateglinide, Z-L-Phe chloromethyl ketone, clemastine fumarate, supercinnamaldehyde, practolol, fluvastatin Na, sulindac, BIO, amorolfine, spectinomucin, sibutramine HCl, nelfinavir mesylate, moroxydine HCl, nicotine ditartrate, trequinsin, meglumine, tizanidine HCl, CGP-74514A hydrochloride, tioconazole, TOVOK™ (afatinib), or kasugamycin.

### CROSS-REFERENCE TO RELATED APPLICATION

This application claims benefit under 35 U.S.C. §119(e) of U.S. Provisional Application No. 61/835,023 filed Jun. 14, 2013 and U.S. Provisional Application No. 61/939,906 filed Feb. 14, 2014, the contents of each of which are incorporated herein by reference in its entirety.

### TECHNICAL FIELD

The disclosure herein relates to compositions and methods for the treatment of mTOR hyperactive related diseases and disorders such as lymphangioleiomyomatosis (LAM) and lymphangioleiomyomatosis in tuberous sclerosis complex (TSC/LAM).

### BACKGROUND

The mammalian target of rapamycin (mTOR) signaling pathway is a major player controlling cell growth and cell division. The kinase, mTOR, is a master regulator of protein synthesis that couples nutrient sensing to cell growth. Defects in the mTOR signaling pathway can result in loss of control in cell growth and cell division. For example, two proteins, hamartin and tuberin, are known to be involved in the control of cell growth and cell division via their effects on the mTOR signaling pathway. Hamartin and tuberin function as a complex to interact with Rheb GTPase, thereby sequestering it from activating mTOR signaling. Mutations at the TSC1 and TSC2 loci which codes for hamartin and tuberin respectively result in the deregulation of the mTOR signaling pathway resulting in increased mTOR signaling. This in turn leads to a loss of control of cell growth and cell division, and subsequently a predisposition to forming tumors.

There are a number of medical conditions that are associated with a deregulation of the mTOR signaling pathway. For example, lymphangioleiomyomatosis (LAM), and tuberous sclerosis complex (TSC). LAM is a rare lung disease that is associated with mutations in the TSC2 locus. It is characterized by the proliferation of abnormal smooth muscle-like cells throughout the lungs, in the bronchioles, alveolar septa, perivascular spaces, and lymphatics, resulting in the obstruction of small airways (leading to pulmonary cyst formation and pneumothorax) and lymphatics (leading to chylous pleural effusion). LAM occurs almost exclusively in women, usually of childbearing age. There are two types of LAM, sporadic LAM and LAM/TSC which is LAM that frequently occurs in patients who have TSC.

The clinical course of patients with LAM shows considerable variation. The disease can progress slowly, but ultimately leads to respiratory failure and death. The 10-year survival rate from the start of symptoms has been reported to range from 47-79% depending on the various studies. Current treatments include administration of rapamycin (also known as sirolimus, an mTOR inhibitor) for shrinking tumors, and therapies targeting the reproductive cycle of the women, e.g., progesterone, oophorectomy, tamoxifen, gonadotropin-releasing hormone (GnRH) agonists or analogues and androgen therapy.

TSC is a rare multi-system genetic disease that results in the growth of nonmalignant tumors in the brain and on other vital organs such as the kidneys, heart, eyes, lungs, brain, and skin. A combination of symptoms may include seizures, developmental delay, behavioral problems, skin abnormalities, and lung and kidney disease. TSC is caused by a mutation of either of two genes, TSC1 and TSC2.

High percentages (60-80%) of TSC patients have benign tumors in the kidneys called angiomyolipomas (AML) which frequently causing hematuria. These tumors are composed of vascular tissue (angio-), smooth muscle (-myo-), and fat (-lipoma). Although benign, AML may grow such that kidney function is impaired or the blood vessels may dilate and burst leading to catastrophic hemorrhage either spontaneously or with minimal trauma. Large AML can be treated with embolization.

In addition, TSC patients who have AML are predisposed to develop LAM in the lungs. The proliferating smooth muscle that occurs in the type of LAM seen in these patients (TSC-LAM) has been shown to represent clones of the smooth muscle in those patients' renal AML. It is believed to represent metastases of this “benign” tumor.

Leading causes of death in TSC patients include renal disease, brain tumor, LAM of the lung, and status epilepticus or bronchopneumonia in those with severe mental handicap. There is no current effective treatment for TSC or the consequential AML or LAM; treatment is mainly symptomatic management, e.g., everolimus (derivative of rapamycin) for the treatment of subependymal giant cell astrocytoma (brain tumor), vigabatrin for infantile spasm, ACTH for epilepsy and rapamycin for shrinking the tumors.

### SUMMARY

The methods and compositions provided herein relate, in part, to the discovery of compounds having cytotoxic or anti-proliferative activity in cells lacking TSC2. Such compounds can be used as single agents for the treatment of mTOR hyperactivity disorders and do not require pre-treatment with rapamycin.

Candidate compounds were screened for cytotoxic or anti-proliferative activity in cells lacking TSC2. Compounds that were more cytotoxic or anti-proliferative in the absence of rapamycin pretreatment were selected for further study. Since rapamycin is a selective inhibitor of mTOR, the cytotoxic or anti-proliferative activity of these compounds requires an active or hyperactive mTOR pathway. This indicates that these compounds will exhibit a wide therapeutic index due to the selectively high mTOR pathway activity in TSC, LAM and certain cancers, compared to normal tissues. The following compounds were identified to have cytotoxic or antiproliferative activity in the absence of rapamycin and include nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin.

These compounds were surprisingly effective at inducing cell death in TSC2-null LAM cells when used individually in the absence of rapamycin. Therefore, nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin, represent additional therapeutics for inducing apoptosis when desired. In some embodiments, at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin can be used in the embodiments described herein.

Accordingly, in one embodiment, it is the objective of this disclosure to provide additional cancer or anti-tumor therapeutics to the existing repertoire of cancer/anti-tumor therapies currently available, particularly for the treatment of cancer(s) associated with mTOR hyperactivation. In some embodiments, the cancers are associated with mutations in the TSC1 and/or TSC2 loci, and/or have deregulated mTOR signaling or mTOR hyperactivity. In one embodiment, the deregulated mTOR signaling results in mTOR hyperactivity.

In one embodiment, the additional cancer or anti-tumor therapeutics are known drugs that are not currently being use for the treatment of cancer, LAM, or TSC.

In one embodiment, it is the objective of this disclosure to provide additional therapeutics for the treatment and/or prevention of LAM, and also for the treatment and/or management of tuberous sclerosis complex (TSC).

In another embodiment, it is also the objective of this disclosure to provide a new use for known drugs, nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™, and kasugamycin, i.e., use for inducing apoptosis, e.g., for cancer/anti-tumor treatment, treatment and/or prevention of LAM, and also for the treatment and/or management of TSC, wherein the cancer, LAM or TSC is associated with mutations in the TSC1 and/or TSC2 loci, and/or have deregulated mTOR signaling or mTOR hyperactivity.

In another embodiment, it is also the objective of this disclosure to provide new compositions for use in inducing apoptosis, for cancer/anti-tumor treatment, treatment and/or prevention of LAM, and also for the treatment and/or management of TSC, wherein the cancer, LAM or TSC is associated with mutations in the TSC1 and/or TSC2 loci, and/or have deregulated mTOR signaling or mTOR hyperactivity.

Cells with detectable levels of mTOR deregulation and/or hyperactivity are known to be associated with a number of diseases, including, but not limited to, cancer; lymphangioleiomyomatosis (LAM); angiomyolipomata (AML); Cowden's disease; Proteus syndrome; Lhermitte-Duclose disease; Peutz-Jeghers syndrome (PJS); familial hypertrophic cardiomyopathy (HCM); prostate cancer; breast cancer; lung cancer; bladder cancer; melanoma; renal cell carcinoma; ovarian cancer; endometrial cancer; thyroid cancer; glioblastoma; chronic myeloid leukemia (CML); and tuberous sclerosis complex (TSC) (see, e.g. Guertin and Sabatini. Trends in Molecular Medicine 2005 37:S25-S30; which is incorporated by reference herein in its entirety).

In one aspect, described herein is a method for inhibiting cell growth, the method comprising contacting a cell with an effective amount of at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin; wherein the cell has a detectable level of mTOR deregulation or hyperactivity.

In one embodiment, the disclosure herein provides a method for treating cancer in a subject comprising administering to a subject in need thereof a therapeutically effective amount of at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin.

In one embodiment, the disclosure herein provides a method for treating cancer in a subject, the method comprising determining whether cancer cells of the subject involves mTOR deregulation or hyperactivity; and, if so, administering to the subject a therapeutically effective amount of at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin.

In one embodiment, the disclosure herein provides a method for treating cancer in a subject comprising administering to a subject in need thereof a therapeutically effective amount of at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™, and kasugamycin, wherein the cancer cells of the subject comprises mTOR deregulation or hyperactivity or is associated with mutations in the TSC1 and/or TSC2 loci.

In one embodiment, the disclosure herein provides a treatment method for TSC in a subject comprising administering to a subject in need thereof a therapeutically effective amount of at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™, and kasugamycin.

In one embodiment of any method described, at least one compound selected from the group consisting nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™, and kasugamycin is administered.

In another embodiment of any method described, more than one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™, and kasugamycin.

In one embodiment of any method described, when one or more compounds are used for treatment, nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™, and/or kasugamycin is singly administered by a route selected from the group consisting of: aerosol, direct injection, local, systemic, intradermal, direct inhalation, intravitreal, intramuscular, intraperitoneal, intravenous, intrathecal, intrapleural, intrauterine, subcutaneous, epidural, topical, oral, transmucosal, buccal, rectal, vaginal, transdermal, intranasal, intrasynovial, intraocular/periocular, intraorgan, intratumor, and parenteral routes of administration. In one embodiment, where more than one compound is used for treatment, the compounds are administered simultaneously. In another embodiment, where more than one compound is used for treatment, the compounds are administered sequentially. The compounds can be admixed prior to administration and administered together, for example, in a single pharmaceutical composition.

In some embodiments, the one or more compounds used for treatment is administering by nasal inhalation such as via a nebulizer. For example, the agent can be formulated as a powder for delivery via a nebulizer.

In one embodiment of any method described, the composition comprising at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™, and kasugamycin is administered singly or in combination by a route selected from the group consisting of: aerosol, direct injection, local, systemic, intradermal, direct inhalation, intravitreal, intramuscular, intraperitoneal, intravenous, intrathecal, intrapleural, intrauterine, subcutaneous, epidural, topical, oral, transmucosal, buccal, rectal, vaginal, transdermal, intranasal, intrasynovial, intraocular/periocular, intraorgan, intratumor, and parenteral administration.

In one embodiment of any method described, nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin are administered in conjunction with at least one additional therapy to achieve a combination therapy.

In another embodiment of any method described, the composition comprising at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin is further administered with a pharmaceutically acceptable carrier.

In one embodiment of any method described herein, the cancer involves mTOR deregulation or hyperactivity. In one embodiment, the mTOR deregulation results in mTOR hyperactivity.

In one embodiment of any method described herein, the cancer involving mTOR deregulation or hyperactivity is LAM. In another embodiment, the cancer in LAM.

In one embodiment of any method, the mTOR hyperactivity is at least 10% higher compared to a control mTOR activity level.

In one embodiment of any method described herein, the control mTOR activity level is an mTOR activity level in a population of normal non-cancer cells of the subject or an average mTOR activity level in a population of healthy subjects.

In one embodiment of any method described herein, a tumor in the subject being treated is reduced in size by at least 10%.

In one embodiment, the subject is treated with at least one additional therapy in addition to treatment with at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin. In one embodiment of any method described herein, the at least one additional therapy is a cancer therapy.

In one embodiment of any method described herein, the at least one additional cancer therapy is selected from the group consisting of radiation therapy, chemotherapy, immunotherapy and gene therapy.

In one embodiment of any method described herein, the subject is human.

In one embodiment of any method described herein, each compound is administered singly, i.e., each compound is administered independently of the others. In another embodiment of any method described, the compounds are administered singly and simultaneously. In another embodiment, the compounds are administered together, e.g., in a cocktail or admixture.

In one embodiment, the disclosure herein provides a composition comprising at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin for use in the treatment of cancer and/or TSC.

In one embodiment, the disclosure herein provides a composition comprising at least two compounds selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin for use in the treatment of cancer and/or TSC. In one embodiment, the composition further comprises a pharmaceutical carrier.

In one embodiment, the disclosure herein provides a composition comprising more than one compound (e.g., 2, 3, 4, 5, 6, 7, or more) selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin for use in the treatment of cancer and/or TSC. In one embodiment, the composition further comprises a pharmaceutical carrier.

In one embodiment of any composition described, the cancer involves mTOR deregulation or hyperactivity. In one embodiment, the mTOR deregulation results in mTOR hyperactivity.

In one embodiment of any composition described, the cancer involving mTOR deregulation or hyperactivity is LAM. In another embodiment, the cancer is LAM.

In one embodiment of any composition described, the mTOR hyperactivity is at least 10% higher compared to a control mTOR activity level.

In one embodiment of any composition described, the control is an mTOR activity level in a population of normal non-cancer cells of the subject or an average mTOR activity level in a population of healthy subjects.

In one embodiment of any composition described, the composition further comprises a pharmaceutically acceptable carrier.

In one embodiment, the subject is treated with at least one additional therapy in addition to treatment with at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin. In one embodiment of any composition described, the at least one additional therapy is a cancer therapy.

In one embodiment of any composition described, the at least one additional cancer therapy is selected from the group consisting of radiation therapy, chemotherapy, immunotherapy and gene therapy.

In one embodiment of any composition described, the composition is formulated for administration by a route selected from the group consisting of: aerosol, direct injection, local, systemic, intradermal, direct inhalation, intravitreal, intramuscular, intraperitoneal, intravenous, intrathecal, intrapleural, intrauterine, subcutaneous, epidural, topical, oral, transmucosal, buccal, rectal, vaginal, transdermal, intranasal, intrasynovial, intraocular/periocular, intraorgan, intratumor, and, and parenteral administration.

In one embodiment of any of the methods or compositions described herein, derivatives or analogues of the known drugs/compounds are included.

Another aspect of the invention relates to the use of one or more compounds selected from the group consisting of nateglinide, Z-L-Phe chloromethyl ketone, clemastine fumarate, supercinnamaldehyde, practolol, fluvastatin Na, sulindac, BIO, amorolfine, spectinomucin, sibutramine HCl, nelfinavir mesylate, moroxydine HCl, nicotine ditartrate, trequinsin, megluime, tizanidine HCl, CGP-74514A hydrochloride, tioconazole, afatinib, and kasugamycin for the preparation of a medicament for the treatment of cancer.

Yet another aspect of the invention relates to the use of one or more compounds selected from the group consisting of nateglinide, Z-L-Phe chloromethyl ketone, clemastine fumarate, supercinnamaldehyde, practolol, fluvastatin Na, sulindac, BIO, amorolfine, spectinomucin, sibutramine HCl, nelfinavir mesylate, moroxydine HCl, nicotine ditartrate, trequinsin, megluime, tizanidine HCl, CGP-74514A hydrochloride, tioconazole, afatinib, and kasugamycin for the preparation of a medicament for the treatment of tuberous sclerosis complex.

### DETAILED DESCRIPTION

The methods and compositions described herein are based, in part, on the discovery of compounds that can be used as single agents for inhibiting the growth of TSC-2 null cells. The compounds described herein are effective when the mTOR pathway is active. Such compounds are useful in treating diseases resulting from mTOR hyperactivation, for example, TSC, LAM and certain cancers.

### DEFINITIONS

As used herein, the term “apoptosis” refers to a natural process of self-destruction in certain cells that is initiated and/or determined by activation of certain genes. Apoptosis can be initiated by an external stimulus e.g., administration of an inducer of apoptosis. Several biochemical events lead to characteristic cell changes (morphology) and death. These changes include, but are not limited to, cell blebbing, cell shrinkage, nuclear fragmentation, chromatin condensation, and chromosomal DNA fragmentation. Analysis of apoptosis can be performed by any method known in the art; non-limiting examples include cell free apoptotic assay, DNA fragmentation assay, DNA laddering assay, terminal transferase dUTP nick end labeling (TUNEL) assay and Annexin A5 (or annexin V) detection. The DNA can be labeled with propidium iodide or 7-AAD and analyzed by flow cytometry.

A “cancer” in a subject refers to the presence of cells possessing characteristics typical of cancer-causing cells, such as uncontrolled proliferation, immortality, metastatic potential, rapid growth and proliferation rate, loss of contact inhibition and certain characteristic morphological features. Often, cancer cells will be in the form of a tumor, but such cells may exist alone within a subject, or may be a non-tumorigenic cancer cell, such as a leukemia cell. Examples of cancer include but are not limited to breast cancer, melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, LAM, leukemia, liver cancer, lung cancer, lymphoma, non-small cell lung cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, TSC, urinary bladder cancer, uterine or endometrial cancer, and vulval cancer.

As used herein, the term “tumor” means a mass of transformed cells that are characterized by neoplastic uncontrolled cell multiplication and at least in part, by containing angiogenic vasculature. The abnormal neoplastic cell growth is rapid and continues even after the stimuli that initiated the new growth has ceased. The term “tumor” is used broadly to include the tumor parenchymal cells as well as the supporting stroma, including the angiogenic blood vessels that infiltrate the tumor parenchymal cell mass. Although a tumor generally is a malignant tumor, i.e., a cancer having the ability to metastasize (i.e., a metastatic tumor), a tumor also can be nonmalignant (i.e., non-metastatic tumor). Tumors are hallmarks of cancer, a neoplastic disease the natural course of which is fatal. Cancer cells exhibit the properties of invasion and metastasis and are highly anaplastic.

As used herein, the term “cancer therapy” refers to a therapy useful in treating cancer. In some embodiments, the cancer therapy involves the use of anti-cancer therapeutic agents and medical procedures. Non-limiting examples of cancer therapy and anti-cancer therapeutic agents include, but are not limited to, e.g., surgery, chemotherapeutic agents, immunotherapy, growth inhibitory agents, cytotoxic agents, agents used in radiation therapy, anti-angiogenesis agents, apoptotic agents, anti-tubulin agents, and other agents to treat cancer, such as anti-HER-2 antibodies (e.g., HERCEPTIN), anti-CD20 antibodies, an epidermal growth factor receptor (EGFR) antagonist (e.g., a tyrosine kinase inhibitor), HER1/EGFR inhibitor (e.g., erlotinib (TARCEVA)), platelet derived growth factor inhibitors (e.g., GLEEVEC™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also contemplated for use with the methods described herein.

In one embodiment, “administration,” “treating,” and “treatment,” as it applies to a subject, refers to the contact of an exogenous pharmaceutical, a drug, a compound, a therapeutic, or a composition to the subject. In another embodiment, “administration,” “treating,” and “treatment,” as it applies to a subject, refers to the contact of any one of the described compounds or compositions to the subject. For example, contacting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™, or kasugamycin with the subject.

Alternatively, the term “administering,” refers to the placement of a compound, a combination of compound, or a composition described herein for intended purposes such as treating cancer, inhibiting cell growth, killing cells or inducing apoptosis, into a subject by a method or route which results in at least partial localization of the compound, the combination of compound, or the composition respectively at a desired site, i.e., cancer cells, tumor cells, tumor cells with TSC mutation(s) and/or mTOR hyperactivity in the subject. The compound, the combination of compounds, or the composition(s) described herein can be administered by any appropriate route which results in effective treatment of the subject, i.e. administration results in delivery to a desired location (e.g., directly to a tumor or near a tumor) in the subject where at least a portion of the composition delivered. The period of time the compound, the combination of compounds, or the composition(s) is/are active depends on the half-life in vivo after administration to a subject, and can be as short as a few hours, e.g., twenty-four hours, to a few days, to as long as several years. Modes of administration include injection, infusion, instillation, suppository (e.g., for vaginal, cervical. rectal or urethral insertion), percutaneous implantation or ingestion. “Injection” includes, without limitation, intravenous, intramuscular, intraarterial, intraventricular, intradermal, intraperitoneal, subcutaneous, subcuticular injection and infusion.

In one embodiment, as used herein, the term “treat” or “treatment” refers to reducing or alleviating at least one adverse clinical symptom associated with cancer, e.g., pain, swelling, tumor size, tumor growth rate, low blood count etc. In another embodiment, the term “treat” or “treatment” refers to slowing or reversing the progression of neoplastic uncontrolled cell multiplication, i.e., shrinking existing tumors and/or halting tumor growth. In another embodiment, the term “treat” or “treatment” refers to inducing apoptosis in cancer or tumor cells in the subject.

In one embodiment, as used herein, the term “prevention” or “preventing” when used in the context of a subject refers to stopping, hindering, and/or slowing down the development of tumors and symptoms associated with aberrant formation of such tumor.

As used herein, the term “a therapeutically effective amount” or “an effective amount” refers to an amount sufficient to achieve the intended purposes such as treating cancer, inhibiting cell growth, killing cells or inducing apoptosis. In one embodiment, a therapeutically effective amount of a compound, a combination of compounds, a pharmaceutical formulation, or a composition described herein for a method of treating cancer or TSC is an amount of sufficient to induce apoptosis of cancer cells of the subject as compared to the level of apoptosis/cell death in the absence of the compound, the combination of compounds, the pharmaceutical composition/formulation or the composition. In other embodiments, the amount of the composition administered is preferably safe and sufficient to treat, delay the development of a tumor, and/or delay further growth of the tumor. In some embodiments, the amount can thus cure or result in amelioration of the symptoms of cancer and tumor growth, slow the course of cancer progression, slow or inhibit a symptom of cancer, slow or inhibit the establishment of secondary symptoms of cancer or inhibit the development of a secondary symptom of the cancer. For example, an effective amount of a compound, a combination of compounds, or a composition described herein inhibits further tumor growth (e.g., LAM or AML), cause a reduction in size or even completely halt tumor growth, shrink the size of a tumor(s), even initiate complete regression of tumor, and reduce clinical symptoms associated with a tumor. In one embodiment, an effective amount for treating cancer or TSC is an amount of a compound, a combination of compounds, or a composition described herein sufficient to result in a reduction or complete removal of the symptoms of the disorder, disease, or medical condition. In another embodiment, an effective amount for treating or ameliorating a disorder, disease, or medical condition is an amount sufficient to result in a reduction or complete removal of the symptoms of the disorder, disease, or medical condition. The effective amount of a given therapeutic agent will vary with factors such as the nature of the agent, the route of administration, the size and species of the animal to receive the therapeutic agent, and the purpose of the administration. Thus, it is not possible or prudent to specify an exact “therapeutically effective amount.” However, for any given case, an appropriate “effective amount” can be determined by a skilled artisan according to established methods in the art using only routine experimentation.

Derivatives, as used herein, include a chemically modified compound wherein the modification is considered routine by the ordinary skilled chemist, such as additional chemical moieties (e.g., an ester or an amide of an acid, protecting groups, such as a benzyl group for an alcohol or thiol, and tert-butoxycarbonyl group for an amine). Derivatives also include radioactively labeled derivatives of the compounds described herein (e.g., biotin or avidin, with enzymes such as horseradish peroxidase and the like, with bioluminescent agents, chemoluminescent agents or fluorescent agents). Additionally, moieties can be added to the compounds described herein or a portion thereof to increase half-life in vivo. Derivatives, as used herein, also encompasses analogs, such as a compound that comprises a chemically modified form of a specific compound or class thereof, and that maintains the pharmaceutical and/or pharmacological activities characteristic of the compound or class, are also contemplated herein. In one embodiment, the term “derivatives”, as used herein, also encompasses prodrugs of the compounds described herein, which are known to enhance numerous desirable qualities of pharmaceuticals (e.g., solubility, bioavailability, manufacturing, etc.).

The term “analogue” or “analog”, as used herein, refers to a chemical compound having a structure similar to that of another but differing from it in respect to a certain component, e.g., it can have a similar action metabolically. In one embodiment, an analog is a drug that is similar to the drug from which it is derived.

As used herein, the terms “drug” and “compound” are used interchangeably and they refer to a known drug described herein.

As used herein, the term “mTOR deregulation” with respect to cancer cells or cells with neoplasia refers to increased or decreased signaling of the mTOR pathway compared to normal cells or cells without neoplasia. Increased or decreased signaling can be analyzed by any method known in the art, e.g., by monitoring the corresponding increase or decrease phosphorylation of the mTOR downstream effectors molecules S6K1 and 4E-BP1. See L. Yan, 2006 J. Biol. Chem., 281: 19793-19797.

As used herein, the term “mTOR hyperactivation” with respect to cancer cells or cells with neoplasia refers to increased signaling of the mTOR pathway compared to normal cells or cells without neoplasia. Increased mTOR signaling can be analyzed by any method known in the art, e.g., by monitoring the increase phosphorylation of the mTOR downstream effectors molecules S6K1 and 4E-BP1. See L. Yan, 2006 J. Biol. Chem., 281: 19793-19797.

As used herein, the term “neoplasia” refers to the abnormal proliferation of benign or malignant cells.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

As used herein the term “cell proliferation” or “cell growth” refers to reproduction and increase in cell number, i.e., cell division.

As used herein in the context of a level of mTOR deregulation or hyperactivity, a “detectable level” refers to a level of deregulation and/or hyperactivity in a sample that allows the regulation and/or activity of mTOR to be distinguished from a reference level, e.g. the regulation and/or activity of mTOR in a reference level (e.g., mTOR activity in a cancer free sample), by at least one of the methods and/or assays for mTOR regulation and/or activity described elsewhere herein. In some embodiments, a detectable level of mTOR hyperactivity can be a level of mTOR activity at least 10% greater than a reference level, e.g. 10% greater, 20% greater, 50% greater, 100% greater, 200% greater, or 300% or greater.

As used herein the term “comprising” or “comprises” is used in reference to compositions, methods, and respective component(s) thereof, that are essential to the disclosure, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of this disclosure.

The term “consisting of” refers to compositions, methods, and respective components thereof as described herein, which are exclusive of any element not recited in that description of the embodiment.

Unless otherwise explained, all technical and scientific terms used herein have the same meaning as commonly understood by one of ordinary skill in the art to which this disclosure belongs. Definitions of common terms in molecular cell biology may be found in Harvey Lodish et al., Molecular Cell Biology, 6^{th} edition, published by W. H. Freeman and Company, 2007 (ISBN 0716776014); Kendrew et al. (eds.), The Encyclopedia of Molecular Biology, published by Blackwell Science Ltd., 1994 (ISBN 0716776014); and Robert A. Meyers (ed.), Molecular Biology and Biotechnology: a Comprehensive Desk Reference, published by VCH Publishers, Inc., 1995 (ISBN 1-56081-569-8). Further, unless otherwise required by context, singular terms shall include pluralities and plural terms shall include the singular.

Unless otherwise stated, the technology and embodiments thereof presented herein can be performed using standard procedures known to one skilled in the art, for example, in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); Current Protocols in Immunology (CPI) (John E. Coligan, et. al., ed. John Wiley and Sons, Inc.), Current Protocols in Cell Biology (CPCB) (Juan S. Bonifacino et. al. ed., John Wiley and Sons, Inc.), Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998), and Methods in Molecular biology, Vol. 180, Transgenesis Techniques by Alan R. Clark editor, second edition, 2002, Humana Press, which are all herein incorporated by reference in their entireties.

It should be understood that this technology is not limited to the particular methodology, protocols, and reagents, etc., described herein and as such may vary. The terminology used herein is for the purpose of describing particular embodiments only, and is not intended to limit the scope of the present technology, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages will mean±1%.

All patents and publications identified are expressly incorporated herein by reference for the purpose of describing and disclosing, for example, the methodologies described in such publications that might be used in connection with the present technology. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Compounds for Treating Diseases of mTOR Hyperactivation

Several known drugs were discovered herein to be effective at inducing cell death in TSC2-null LAM cells in the absence of rapamycin. The drugs are nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™, and kasugamycin, Therefore, these known drugs represent therapeutics for inducing apoptosis when desired.

Accordingly, in one embodiment, provided herein is a method of inhibiting the growth of a cell comprising contacting the cell with an effective amount with an effective amount of at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin, wherein the effective amount is effective to inhibit growth of the cell.

In one embodiment, provided herein is a method of inducing apoptosis in a cell comprising contacting the cell with an effective amount of at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin, wherein the effective amount is effective to induce apoptosis in the cell.

In one embodiment of any method described herein, for example, for inhibiting cell growth, killing of cells or inducing apoptosis in cells, the cell is a cancer cell. For example, a bladder cancer cell, a blood cancer, a breast cancer cell, a lung cancer cell, a colon cancer cell, a prostate cancer cell, a liver cancer cell, a pancreatic cancer cell, a stomach cancer cell, a testicular cancer cell, a brain cancer cell, an ovarian cancer cell, a lymphatic cancer cell, a skin cancer cell, a brain cancer cell, a bone cancer cell, a soft tissue cancer cell, among others.

In another embodiment of any method of inhibiting cell growth, killing of cells or inducing apoptosis in cells, the cell is a tumor cell.

In one embodiment of any method of inhibiting cell growth, killing of cells or inducing apoptosis in cells, the cell is located in a subject. In one embodiment, the subject is a human subject.

In one embodiment of any method of inhibiting cell growth, killing of cells or inducing apoptosis in cells, the cell has a mutation at the TSC1 and/or TSC2 locus.

In one embodiment of any method of inhibiting cell growth, killing of cells or inducing apoptosis in cells, the cell has mTOR deregulation or mTOR hyperactivity. In one embodiment, the mTOR deregulation is mTOR hyperactivity.

In one embodiment of any method of inhibiting cell growth, killing of cells or inducing apoptosis in cells, the at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin is administered by direct intratumoral injection.

In one embodiment of any method of inhibiting cell growth, killing of cells or inducing apoptosis in cells, the at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin is administered by injection into tumor vasculature.

In one embodiment of any method of inhibiting cell growth, killing of cells or inducing apoptosis in cells, the at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin is about 0.5 mg/kg to about 10 mg/kg.

In one embodiment of any method of inhibiting cell growth, killing of cells or inducing apoptosis in cells, the at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin is about 1 mg/kg to about 4 mg/kg.

In one embodiment of any method of inhibiting cell growth, killing of cells or inducing apoptosis in cells, the at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin is administered in combination with at least one additional agent. In one embodiment of any method of inhibiting cell growth, killing of cells or inducing apoptosis in cells, the at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin and the at least one additional agent are administered individually.

In one embodiment of any method of inhibiting cell growth, killing of cells or inducing apoptosis in cells, the at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin and the at least one additional agent are administered simultaneously.

In one embodiment of any method of inhibiting cell growth, killing of cells or inducing apoptosis in cells, the individual contacting of the at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin and the contacting of the at least one additional agent occurs sequentially.

In one embodiment of any method of inhibiting cell growth, killing of cells or inducing apoptosis in cells, the at least one additional agent and the at least one compound selected from the group consisting nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin are admixed in a cocktail or a pharmaceutical composition prior to contacting with the cell or administration.

In one embodiment of any method of inhibiting cell growth, killing of cells or inducing apoptosis in cells, when more than one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin are used, the compounds are individually contacted with the cell.

In one embodiment of any method of inhibiting cell growth, killing of cells or inducing apoptosis in cells, when more than one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin are used and the compounds are individually contacted with the cell, the individual contacting of the compounds occurs simultaneously.

In one embodiment of any method of inhibiting cell growth, killing of cells or inducing apoptosis in cells, when more than one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin are used and the compounds are individually contacted with the cell, the individual contacting of the compounds occurs sequentially.

In one embodiment of any method of inhibiting cell growth, killing of cells or inducing apoptosis in cells, when more than one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin are used, the compounds are admixed in a cocktail or as a composition prior to contacting with the cell.

In one embodiment, provided herein is a method for treating cancer in a subject comprising administering to a subject in need thereof a therapeutically effective amount of at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin.

In one embodiment, provided herein is a method for treating cancer in a subject, the method comprising determining whether cancer cells of the subject involves mTOR deregulation or hyperactivity; and, if so, administering to the subject a therapeutically effective amount of at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin.

In one embodiment, provided herein is a method for treating cancer in a subject comprising administering to a subject in need thereof a therapeutically effective amount of at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin, wherein the cancer involves mTOR deregulation or hyperactivity.

In one embodiment, provided herein is a method for treating TSC in a subject comprising administering to a subject in need thereof a therapeutically effective amount of at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin.

In one embodiment, for any method of treating cancer as described herein, the method further comprises a step of selecting a subject having cancer (e.g., imaging, tumor biopsy, endoscopic examination, surgery, genetic testing or laboratory tests e.g., measuring blood urea nitrogen levels (BUN), complete blood count (CBC), urinalysis for hematuria or proteinuria, or tumor markers such as prostate specific antigen (PSA), prostatic acid phosphatase (PAP), CA125, carcinoembryonic antigen (CEA), alpha fetoprotein (AFP), human chorionic gonadotropin (HCG), CA 19-9, CA 15-3, CA 27-29, lactate dehydrogenase (LDH), or neuron-specific enolase (NSE)).

In one embodiment, for any method of treating TSC as described herein, the method further comprises a step of selecting a subject having cancer (e.g., detecting a genetic mutation in TSC1 and/or TSC2).

In one embodiment, for any method of treating mTOR deregulation and/or mTOR hyperactivity, the method further comprises a step of selecting a subject having mTOR deregulation and/or mTOR hyperactivity (e.g., in patients refractory at least one other cancer treatment, by detecting elevated Akt activity, by detecting PI3KCA or PTEN mutations, or by detecting an mTOR polymorphism).

mTOR Signaling Pathway

The mTOR signaling pathway is a major player in controlling cell growth and cell division. Cancers associated with genetic defects often have aberrant mTOR signaling. The inventors have discovered that known drugs are effective at inducing apoptosis in LAM cells having TSC mutations. LAM is caused by mutations in TSC2, which encodes the protein tuberin (TSC2). The TSC1/TSC2 heterodimer, through inhibition of the Ras homolog enriched in the brain protein (Rheb), negatively regulates the mammalian target of rapamycin (mTOR) complex 1 (TORC1). Therefore, LAM patient lesions have hyperactivation of TORC1. Rapamycin is a naturally occurring macrolide that inhibits TORC1 actively and is effective in shrinking kidney angio myolipomas (AML). Therefore, these known drugs are also effective at inducing apoptosis in cells having deregulated mTOR pathway signaling, and mTOR hyperactivity.

Accordingly, in one embodiment of any method described, the contacted cell or cancer to be treated involves mutations in at least one of the TSC loci. In one embodiment, the mutation is at the TSC1 locus. In another embodiment, the mutation is at the TSC2 locus. In another embodiment, the mutation is at both the TSC1 and TSC2 loci.

In one embodiment of any method described, the contacted cell or the cancer to be treated involves mTOR deregulation or hyperactivity. In one embodiment, the mTOR deregulation results in mTOR hyperactivity.

In one embodiment of any method, the mTOR hyperactivity is at least 10% higher compared to a control mTOR activity level. In other embodiments, the mTOR hyperactivity is at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100% over the mTOR control. In other embodiment, the mTOR activity level is at least 2-fold, at least 5-fold, at least 10-fold, at least 100-fold, at least 1000-fold or higher as compared to the control mTOR activity level.

In one embodiment of any method described herein, the mTOR control is an mTOR activity level in a population of normal non-cancerous cells from the subject being treated. In another embodiment, the mTOR control is an average mTOR activity level in a population of healthy subjects. For example, normal non-cancer cells can be taken from the subject being treated and analyzed for cellular mTOR activity level. The normal non-cancer cells can be taken from the same organ diagnosed with cancer or tumors, or the normal non-cancer cells can be taken from other healthy organs that are free from cancer or tumors in the subject to be treated.

Alternatively, healthy cells can be collected from a population of healthy subjects, e.g., human subjects, the mTOR activity for the cells of each subject is analyzed and the average mTOR activity is calculated. The healthy cells collected from the healthy subjects can be from the same organ where cancer or tumors are diagnosed in the subject being treated. Alternatively, the healthy cells can come from a variety of tissue types in a subject.

For analyzing TSC mutation and/or mTOR activity, a tissue sample is collected from the subject to be treated or healthy volunteer subjects. Cancer cells can be obtained from a subject diagnosed with or suspected of having cancer and/or tumors. For example, cancer cells can be obtained from a tissue biopsy or an excised tumor during a routine surgery to remove cancerous tumors. During the biopsy, healthy, normal non-cancer cells can be taken for analyzing the control cellular mTOR level. A skilled physician or surgeon will be able to obtain a tissue biopsy or excise a tumor from a subject. Alternatively, for TSC gene analysis, a sample of blood from the subject can be used.

In one embodiment, the tissue sample is a tumor sample. In another embodiment, the tissue sample contains cancerous cells.

As used herein, a “tissue sample” refers to a portion, piece, part, segment, or fraction of a tissue which is obtained or removed from an intact tissue of a subject, preferably a human subject. In one embodiment, the tissue sample is a blood sample. In another embodiment, the tissue sample is a bone marrow sample. In one embodiment, the tissue sample is a cerebrospinal fluid sample.

As used herein, a “tumor sample” refers to a portion, piece, part, segment, or fraction of a tumor, for example, a tumor which is obtained or removed from a subject (e.g., removed or extracted from a tissue of a subject), preferably a human subject.

In one embodiment, the tissue sample is obtained from a biopsy procedure in the subject. In another embodiment, the tissue sample is obtained from a surgical procedure to remove a tumor mass from the subject.

The cellular mTOR activity level of cancer cell and normal non-cancer cells can be analyzed by any method known in the art, for example, as described by Ikenoue T. et a., Methods Enzymol. 2009; 452:165-80; and by Jinhee Kim, et al., Methods in Molecular Biology; 2012; 821:215-225. These references are incorporated herein by reference in their entirety. Alternatively, the cellular mTOR activity level can be determined by using any one of the commercially available kits following the manufacturer's protocol, for example, the K-LISA™ mTOR Activity Kit by Merck Millipore Catalogs# CBA055 and CBA104).

For TSC loci gene analysis, the mutations in the TSC loci can be analyzed by any known genomic method in the art. For example, by single-strand conformation polymorphism analysis (SSCP) coupled with DNA sequencing as described by Galina D. et al., Am. J. Respir. Crit. Care Med.; 2001; 163:253-258; Hornigold N, et al., Oncogene; 1999; 18:2657-2661. Briefly, the coding exons of TSC1 or 2 are amplified by polymerase chain reaction (PCR) and the amplified PCR products are then analyzed for variation on DNA gels without glycerol and with 5% glycerol. As a good number of TSC loci mutations result in chain-terminating, quantitative real-time (RT-PCR) assays can be used to analyze the amount of TSC1/2 mRNA as described in Kwiatkowska J. et al., Ann Hum Genet. 1998; 62:277-85. Alternatively, commercial kits are available, e.g., RT² qPCR Primer Assay for Human TSC1 and TSC2 respectively from SABIOSCIENCES™ catalog# PPH00244B-200 and PPH00245F. The PCR primers for the human TSC1 and TSC2 can be purchased from BIORAD. Alternatively, one skilled in the art can design PCR primers for the human TSC1 and TSC2 with the following information regarding the human TSC1 and TSC2 genes:

The gene symbol, TSC1 stands for the gene name tuberous sclerosis 1. Aliases for TSC1 include; KIAA0243, LAM, MGC86987, and TSC. The RefSeqs of TSC1 are NC_000009.11; NG_012386.1; NT_035014.4. Ensembl: ENSG00000165699; Entrez: 7248; UniGene: Hs.370854.

The gene symbol, TSC2, stands for the gene name tuberous sclerosis 2. Aliases for TSC1 include F1143106, LAM, and TSC4. The RefSeqs of TSC2 are: NC_000016.9; NG_005895.1; NG_008412.1; NG_008617.1; and NT_010393.16. Ensembl: ENSG00000103197; Entrez: 7249; UniGene: Hs.90303.

In one embodiment of any method described, the contacted cell or the cancer to be treated involving mTOR deregulation or hyperactivity is LAM. In another embodiment, the cancer is LAM, e.g., the cancer cells tested positive for mTOR hyperactivity or increased mTOR pathway signaling.

Contacting a Cell with a Composition as Described Herein

In one embodiment, the contacting period is at least one hour. In one embodiment, the contact period is at least one hour to 24 hours. In other embodiments, the contact period is at least two, at least three, at least four, at least five, at least six, at least seven, at least eight, at least nine, at least 10, at least 11, at least 12, at least 13, at least 14, at least 15, at least 16, at least 17, at least 18, at least 19, at least 20, at least 21, at least 22, at least 23, or at least 24 hours. In one embodiment, the contact period is between one hour and 24 hours. In other embodiments, the contact period is two, three, four, five, six, seven, eight, nine, 10, 11, 12, 13, 14, 15, 16, 17, 18, 19, 20, 21, 22, 23, or 24 hours, including all the time periods between one to 24 hours to the minute. In other embodiments, the contacting period is between 24-72 hrs, including all the time periods between 24-72 hours to the half hour.

In one embodiment of any method described herein, a tumor in the subject being administered with the respective drugs or drug combinations is reduced in size by at least 10% compared to the tumor size prior to treatment with the respective drugs or drug combinations. In other embodiments, the tumor is reduced in size by at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at even 100% (i.e., below detectable limits) compared to the tumor size prior to treatment with the respective drugs or drug combinations.

In one embodiment, the cell is further contacted with an inhibitor of the mTOR pathway, for example, rapamycin, temsirolimus, everolimus, ridaforolimus, epigallocatechin gallate (EGCG), caffeine, curcumin, resveratrol etc.

### Compounds

Z-L-PHE chloromethyl ketone (ZPCK) is a ketone containing compound that has bioactivity in the NFAT and STAT signaling pathways. Z-L-PHE chloromethyl ketone is also known in the art as L-Carbobenzyloxyphenylalanyl chloromethyl ketone, N-Carbobenzoxy-L-phenylalanyl-chloromethyl ketone, or N-((Benzyloxy)carbonyl)-L-phenylalanine chloromethyl ketone. In one embodiment, Z-L-PHE chloromethyl ketone has the structure of Formula I.

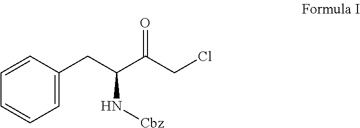 [SMILES: O=C(N[C@@H](Cc1ccccc1)C(=O)CCl)OCc1ccccc1]

Clemastine fumarate is an antihistamine and anticholinergic drug used for the treatment of allergy symptoms, for example, sneezing, watery eyes, itching, wheezing etc. Clemastine fumarate is also known in the art as meclastin, AGASTEN™, TAVEGIL™, and TAVEGYL™. Clemastine fumarate can be obtained commercially from e.g., NOVARTIS™ and TOCRIS BIOSCIENCE™. In one embodiment, clemastine fumarate has the structure of Formula II.

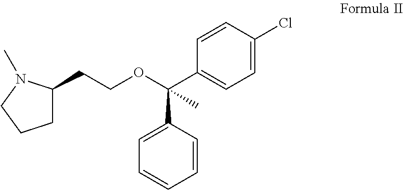 [SMILES: CN1CCC[C@@H]1CCO[C@](C)(c1ccccc1)c1ccc(Cl)cc1]

Supercinnamaldehyde is an organic compound derived from cinnamon and that gives cinnamon its flavor and color. It is used as a flavoring, a fungicide, an insecticide, an antimicrobial and an anti-cancer agent. Supercinnamaldehyde is also known in the art as 1,3-Dihydro-1-methyl-3-(2-oxopropylidene)-2H-Indol-2-one, cinnamaldehyde, trans-Cinnamaldehyde, Cinnamic aldehyde, Cinnamal, (E)-Cinnamaldehyde, 3-Phenylacrylaldehyde, Cinnamylaldehyde, Phenylacrolein, Zimtaldehyde, and Cassia aldehyde, among others. In one embodiment, supercinnamaldehyde has the structure of Formula III.

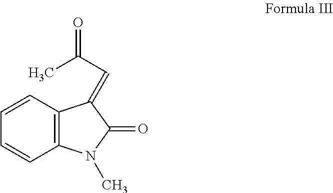

Practolol is a selective beta blocker used in the emergency treatment of cardiac arrhythmias. Practolol is also known in the art as ERALDIN™, DALZIC™, PRAKTOL™, CARDIOL™, PRALON™, CORDIALINA™, ERALDINA™, and TERANO™. In one embodiment, practolol has the formula of Formula IV.

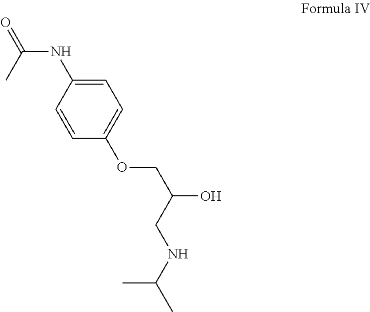 [SMILES: CC(=O)Nc1ccc(OCC(O)CNC(C)C)cc1]

Fluvastatin (fluvastatin Na or fluvastatin sodium) is a member of the statin family of compounds used for the treatment of hypercholesterolemia and the prevention of heart disease. Fluvastatin is also known in the art as LESCOL™, CANEF™, and VASTIN™. In one embodiment, fluvasatin has the formula of Formula V.

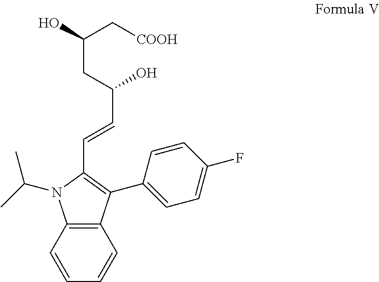 [SMILES: CC(C)n1c(/C=C/[C@@H](O)C[C@@H](O)CC(=O)O)c(-c2ccc(F)cc2)c2ccccc21]

Sulindac is a Non-Steroidal Anti-Inflammatory Drug (NSAID) used for the treatment of pain and inflammatory conditions, such as rheumatoid arthritis, osteoarthritis, gout, dysmenorrhea, metastatic bone pain, fever, headache, muscle stiffness and migraine, among others. Sulindac can be obtained from MERCK™ under the name CLINORIL™. In one embodiment, sulindac has the formula of Formula VI.

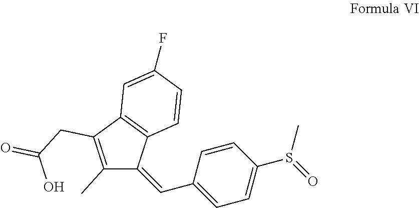 [SMILES: CC1=C(CC(=O)O)c2cc(F)ccc2/C1=C\c1ccc(S(C)=O)cc1]

Amorolfine is a morpholine antifungal drug that is used primarily to treat fungal infections in toenails and fingernails. Amorolfine is also known as CURANAIL™, LOCERYL™, LOCETAR™, and ODENIL™. In one embodiment, amorolfine has the formula of Formula VII.

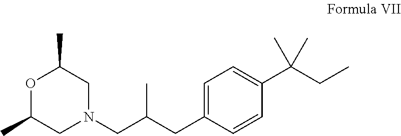 [SMILES: CCC(C)(C)c1ccc(CC(C)CN2C[C@@H](C)O[C@@H](C)C2)cc1]

Spectinomycin is an antibiotic closely related to the aminoglycoside antibiotics. Spectinomycin has been used as an injected antibiotic for the treatment of gonorrhea. Spectinomycin is also known in the art as TROBICIN™. In one embodiment, spectinomycin has the formula of Formula VIII.

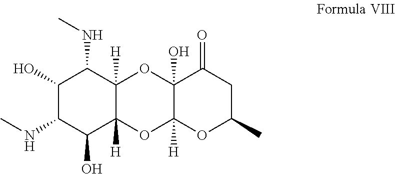

Sibutramine HCL or sibutramine is an oral anorexient used for the treatment of obesity in conjunction with diet and exercise. Sibutramine is also known in the art as REDUCTIL™, MERIDIA™ and SIBUTREX™. In one embodiment, sibutramine has the formula of Formula IX.

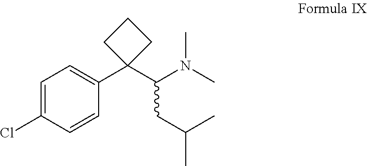 [SMILES: CC(C)CC(N(C)C)C1(c2ccc(Cl)cc2)CCC1]

Nelfinavir mesylate is a protease inhibitor and antiretroviral drug useful for treating HIV infection. Nelfinavir is also known in the art as VIRACEPT™. In one embodiment, nelfinavir has the formula of Formula X.

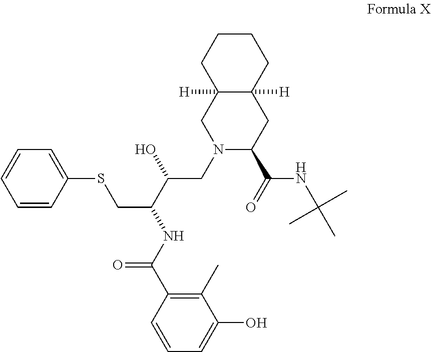 [SMILES: Cc1c(O)cccc1C(=O)N[C@H](CSc1ccccc1)[C@H](O)CN1C[C@H]2CCCC[C@H]2C[C@H]1C(=O)NC(C)(C)C]

Moroxydine HCL or moroxydine is a heterocyclic biguanidine antiviral drug useful for the treatment of influenza. Moroxydine is also known by the following chemical synonyms: ABOB, VIRONIL™, moroxydine; moroxydinum, TIMTEC-BB, SBB003847, 1-(1-morpholinoformimidoyl)guanidine, n-(aminoiminomethyl)-4-morpholinecarboximidamide; 4-morpholinecarboximidamide, and N-(aminoiminomethyl)-; N-[amino(imino)methyl]morpholine-4-carboximidamide hydrochloride. In one embodiment, moroxydine has the formula of Formula XI.

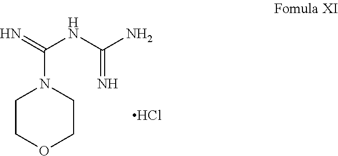 [SMILES: N=C(N)NC(=N)N1CCOCC1 ; Cl]

Nicotine ditartrate is a nicotine derivative that exhibits vasoconstrictive, hypertensive and prothrombotic activity. Nicotine ditartrate is also known by the following chemical synonyms: nicotine tartrate, tartratedenicotine, nicotine bitartrate, nicotine acid tartrate, nicotine, tartrate (1:2), (−)-Nicotine dirartate, and nicotinehydrogentartrate. In one embodiment, nicotine ditartrate has the formula of Formula XII.

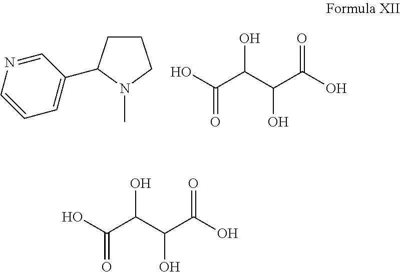

Trequinsin (also known as 9,10-Dimethoxy-2-mesitylimino-3-methyl-2,3,6,7-tetrahydro-4H-pyrimido-[6,1a]-isoquinolin-4-one) is a cGMP-inhibited phosphodiesterase III inhibitor useful in the treatment of short term cardiac failure and intermittent claudication. In one embodiment, trequinsin has the formula of Formula XIII.

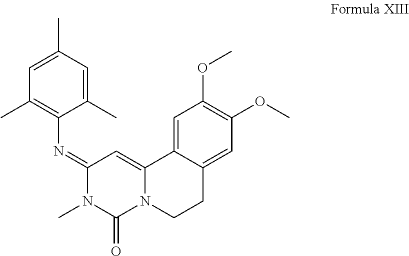

Meglumine is a sugar derived from sorbitol and is typically used as an excipient in pharmaceutical compositions or with iodinated organic compounds as a contrast medium. Meglumine is also known in the art as: N-Methyl-D-glucamine, Meglumin, N-Methylglucamine, 6284-40-8, 1-Deoxy-1-methylaminosorbitol, Megluminum, Methylglucamin, Meglumina, and 1-Deoxy-1-(methylamino)-D-glucitol. In one embodiment, meglumine has the formula of Formula XIV.

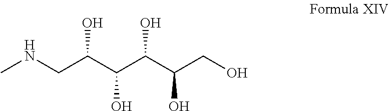

Tizanidine HCl or tizanidine is used as a muscle relaxant in the treatment of a variety of conditions including, but not limited to, spasms, cramping, and tightness of muscles caused by medical problems such as multiple sclerosis, spastic diplegia, back pain, or injuries to the spine or central nervous system. Tizanidine is also known in the art as ZANAFLEX™, and SIRDALUD™. In one embodiment, tizanidine has the formula of Formula XV.

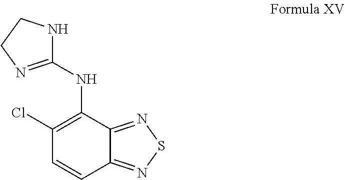 [SMILES: Clc1ccc2nsnc2c1NC1=NCCN1]

CGP-74514A hydrochloride is also known in the art by the following chemical synonyms: compound 13 hydrochloride, and N2-(CIS-2-AMINOCYCLOHEXYL)-N6-(3-CHLOROPHENYL)-9-ETHYL-9H-PURINE-2,6-DIAMINE HYDROCHLORIDE. In one embodiment, CGP-74514A hydrochloride has the formula of Formula XVI.

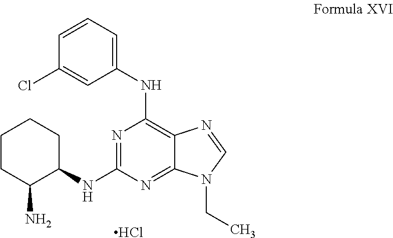 [SMILES: CCn1cnc2c(Nc3cccc(Cl)c3)nc(N[C@@H]3CCCC[C@@H]3N)nc21 ; Cl]

Tioconazole is an antifungal compound useful for treating vaginal yeast infections, ringworm, jock itch, athlete's foot, and tinea versicolor. Tioconazole is also known in the art as TROSYD™, and GYNO-TROSYD™, both of which can be obtained commercially from PFIZER™. In one embodiment, tioconazole has the formula of Formula XVII.

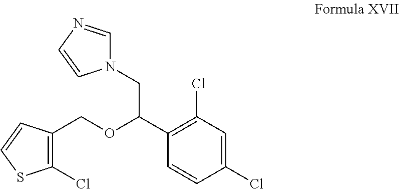 [SMILES: Clc1ccc(C(Cn2ccnc2)OCc2ccsc2Cl)c(Cl)c1]

TOVOK™ (afatinib) is a next generation tyrosine kinase inhibitor and an anti-cancer compound that may be useful in the treatment of non-small cell lung carcinoma, breast cancer, prostate cancer, glioma, and head and neck cancer. TOVOK™ is also known in the art as TOMTOVOK™ and can be obtained commercially from BOEHRINGER INGELHEIM™. In one embodiment, TOVOK™ has the formula of Formula XVIII.

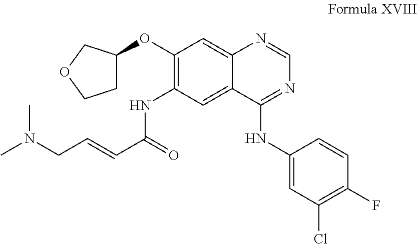 [SMILES: CN(C)C/C=C/C(=O)Nc1cc2c(Nc3ccc(F)c(Cl)c3)ncnc2cc1O[C@H]1CCOC1]

Kasugamycin is an aminoglycoside antibiotic also known as kasumin, 3-O-[2-Amino-4-[(carboxyiminomethyl)amino]-2,3,4,6-tetradeoxy-D-arabino-hexopyranosyl]-D-chiro-inositol, and 2-amino-2-[(2R,3S,5S,6R)-5-amino-2-methyl-6-[(2R,3 S,5S,6S)-2,3,4,5,6-pentahydroxycyclohexyl]oxyoxan-3-yl]iminoacetic acid. In one embodiment, kasugamycin has the formula of Formula XIX.

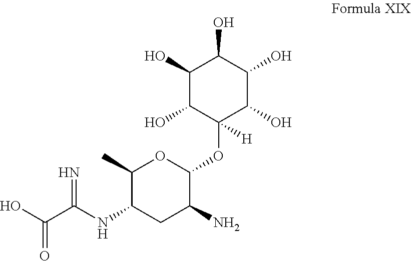 [SMILES: C[C@H]1O[C@H](O[C@H]2[C@H](O)[C@@H](O)[C@@H](O)[C@H](O)[C@@H]2O)[C@@H](N)C[C@@H]1NC(=N)C(=O)O]

Nateglinide is a meglitinide compound used for reducing blood glucose levels in the treatment of Type II diabetes. Nateglinide is also known in the art as STARLIX™ and can be obtained commercially from NOVARTIS™. In one embodiment, nateglinide has the structure of Formula XX.

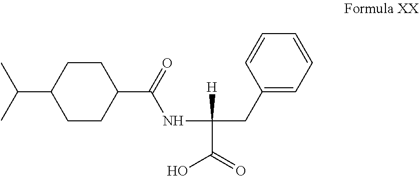 [SMILES: CC(C)C1CCC(C(=O)N[C@H](Cc2ccccc2)C(=O)O)CC1]

Methods for synthesizing the foregoing compounds are known in the art. Moreover, the foregoing compounds are commercially available, e.g. clemastine fumarate is available from NOVARTIS™ as MECLASTIN™.

It is also contemplated that the methods described herein can be used as prophylaxis. Since subjects with TSC are prone to developing tumors in various organs, administration of the described drugs or drug combinations can help prevent tumor formation and thereby reduce the frequency of these tumors in such individuals.

Accordingly, in one embodiment, provided herein is a method of preventing tumor formation in a subject, comprising administering to a subject in need thereof a therapeutically effective amount at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (AFATINIB), and kasugamycin. In one embodiment, the subject has TSC. In one embodiment, the subject has a genetic mutation in at least one of the TSC loci, TSC1 or TSC2. In another embodiment, the method further comprises diagnosing whether the subject has TSC, or has a genetic mutation in at least one of the TSC loci, TSC1 or TSC2.

In one embodiment, provided herein is a method of preventing tumor formation in a subject, comprising determining whether the subject has TSC, or has a genetic mutation in at least one of the TSC loci, TSC1 or TSC2; and, if so, administering to the subject a therapeutically effective amount at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™, and kasugamycin.

In one embodiment, provided herein is a method of reducing the frequency of tumor development in a subject comprising administering to a subject in need thereof a therapeutically effective amount at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™, and kasugamycin. In one embodiment, the subject has TSC. In one embodiment, the subject has a genetic mutation in at least one of the TSC loci, TSC1 or TSC2. In another embodiment, the method further comprises diagnosing whether the subject has TSC, or has a genetic mutation in at least one of the TSC loci, TSC1 and/or TSC2.

In one embodiment, provided herein is a method of reducing the frequency of tumor development in a subject comprising determining whether the subject has TSC, or has a genetic mutation in at least one of the TSC loci, TSC1 and/or TSC2; and, if so, administering to the subject a therapeutically effective amount at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin.

A skilled physician will be able to diagnose TSC based on known clinical symptoms and genetic analysis of the TSC loci in the subject.

In one embodiment, provided herein is a composition comprising at least one compound (e.g., 2, 3, 4, 5, 6, 7, or more) selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin for use in any of the methods described herein, e.g., treatment of cancer and/or TSC, prevention of tumor formation, reducing the frequency of tumor development, inducing apoptosis in a cell, killing a cell and inhibiting cell growth.

In one embodiment, the method for treating cancer in a subject comprises administering to a subject in need thereof a therapeutically effective amount of a composition comprising at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin. In one such embodiment, the cancer comprises mTOR deregulation or hyperactivity.

In one embodiment, provided herein is a method for treating cancer in a subject, the method comprising determining whether cancer cells of the subject comprises mTOR deregulation or hyperactivity; and, if so, administering to the subject a therapeutically effective amount of a composition comprising at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin.

In one embodiment of any method or composition described herein, at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin is administered in combination with at least one additional agent. In one embodiment, the at least one additional agent is a compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin. In such embodiments relating to combination therapy, the compounds or agents can be administered singly, i.e. each compound is administered independently of the others. In another embodiment relating to combination therapy with more than one agent, the agents are administered together, e.g., in a cocktail, admixture or a single pharmaceutical composition.

In one embodiment of any method or composition described herein, the compound(s) are administered by a route selected from the group consisting of aerosol, direct injection, local, systemic, intradermal, direct inhalation, intravitreal, intramuscular, intraperitoneal, intravenous, intrathecal, intrapleural, intrauterine, subcutaneous, epidural, topical, oral, transmucosal, buccal, rectal, vaginal, transdermal, intranasal, intrasynovial, intraocular/periocular, intraorgan, intratumor, and, and parenteral route.

In another embodiment of any method or composition described herein, the composition is administered in conjunction with at least one additional therapy to achieve a combination therapy.

In one embodiment of any method or composition described herein, the composition or the combination of compounds are further administered with a pharmaceutically acceptable carrier.

In one embodiment, provided herein is a composition comprising at least one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin for use in any of the methods described herein, e.g., treatment of cancer and/or TSC, prevention of tumor formation, reducing the frequency of tumor development, inducing apoptosis in a cell, killing a cell and inhibiting cell growth.

In one embodiment, provided herein is a composition comprising more than one compound (e.g., 2, 3, 4, 5, 6, 7, or more) selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin for use in any of the methods described herein, e.g., treatment of cancer and/or TSC, prevention of tumor formation, reducing the frequency of tumor development, inducing apoptosis in a cell, killing a cell and inhibiting cell growth.

In one embodiment of any method or composition described, only one compound selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin is administered. For example, only nateglinide, Z-L-Phe chloroethyl ketone is administered.

In one embodiment of any method or composition described, at least two compounds selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin are administered. In another embodiment of any method or composition described, only two compounds selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin are administered. For example, only practolol and fluvastatin Na are administered. All possible two compound combinations derived from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin are contemplated herein for use as a combination therapy.

In one embodiment of any method or composition described, at least three compounds selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin are administered. In another embodiment of any method or composition described, only three compounds selected from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin are administered. All possible three compound combinations derived from the group consisting of nateglinide, Z-L-Phe chloroethyl ketone, clemastine fumarate, supercinnamaldehyde, practolol, fluvastatin Na, sulindac, BIO, amorolfine, spectinomycin, sibutramine HCl, nelfinavir mesylate, moroxydine HCl, nicotine ditartrate, trequinsin, meglumine, tizanidine HCl, CGP-7451A hydrochloride, tioconazole, TOVOK™ (afatinib), and kasugamycin are contemplated herein for use as a combination therapy.

In one embodiment of any composition described herein, the composition is administered by a route selected from the group consisting of: aerosol, direct injection, local, systemic, intradermal, direct inhalation, intravitreal, intramuscular, intraperitoneal, intravenous, intrathecal, intrapleural, intrauterine, subcutaneous, epidural, topical, oral, transmucosal, buccal, rectal, vaginal, transdermal, intranasal, intrasynovial, intraocular/periocular, intraorgan, intratumor, and, and parenteral administration.

In one embodiment of any method or composition described herein, the compound(s) or composition is administered in conjunction with at least one additional therapy to achieve a combination therapy.

In one embodiment of any method or composition described herein, the compound(s) or composition is further administered with a pharmaceutically acceptable carrier.

In one embodiment of any method or composition described, the at least one additional therapy is a therapy that help the subject cope with cancer treatment side effects. For example, aromatherapy, exercise, hypnosis, massage, meditation, tai chi, yoga, acupuncture, music therapy and relaxation techniques.

In one embodiment of any method or composition described herein, the at least one additional cancer therapy is selected from therapies such as one or more anti-cancer therapeutic agents selected from the group consisting of growth inhibitory agents, cytotoxic agents, anti-angiogenesis agents, apoptotic agents, anti-tubulin agents, anti-HER-2 antibodies, anti-CD20 antibodies, an epidermal growth factor receptor (EGFR) antagonist, a HER1/EGFR inhibitor, a platelet derived growth factor inhibitor, a COX-2 inhibitor, an interferon, and a cytokine (e.g., G-CSF, granulocyte-colony stimulating factor).

In one embodiment of any method or composition described, the at least one additional therapy is a cancer therapy. Non-limiting examples of anti-cancer therapeutic agents are 13-cis-retinoic acid, 2-CdA, 2-Chlorodeoxyadenosine, 5-Azacitidine, azacytidine, 5-Fluorouracil, 5-FU, 6-Mercaptopurine, 6-MP, 6-TG, 6-Thioguanine, abiraterone acetate, Abraxane, Accutane®, Actinomycin-D, Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Arzerra™, Asparaginase, ATRA, Avastin®, Axitinib, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, C225, Cabazitaxel, Calcium Leucovorin, Campath® Camptosar® Camptothecin-11, Capecitabine, Caprelsa® Carac™ Carboplatin, Carmustine, Carmustine Wafer, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Crizotinib, Cyclophosphamide, Cytadren®, Cytarabine, Cytarabine Liposomal, Cytosar-U®, Cytoxan®, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin Diftitox, Denosumab, DepoCyt™, Dexamethasone, Dexamethasone Acetate, Dexamethasone Sodium Phosphate, Dexasone, Dexrazoxane, DHAD, DIC, Diodex, Docetaxel, Doxil®, Doxorubicin, Doxorubicin Liposomal, Droxia™, DTIC, DTIC-Dome®, Duralone®, Eculizumab, Efudex®, Eligard™, Ellence™, Eloxatin™, Elspar®, Emcyt®, Epirubicin, Epoetin Alpha, Erbitux, Eribulin, Erlotinib, Erwinia L-asparaginase, Estramustine, Ethyol, Etopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, Gefitinib, Gemcitabine, Gemtuzumab ozogamicin, Gemzar, Gleevec™, Gliadel® Wafer, Goserelin, Granulocyte-Colony Stimulating Factor (G-CSF), Granulocyte Macrophage Colony Stimulating Factor (GM-CSF), Halaven®, Halotestin®, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetan, Idamycin®, Idarubicin, Ifex®, IFN-alpha, Ifosfamide, IL-11, IL-2, Imatinib mesylate, Imidazole Carboxamide, Inlyta®, Interferon alpha, Interferon Alpha-2b (PEG Conjugate), Interleukin-2, Interleukin-11, Intron A® (interferon alpha-2b), Ipilimumab, Iressa®, Irinotecan, Isotretinoin, Ixabepilone, Ixempra™, Jevtana®, Kidrolase (t), Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilotinib, Nilutamide, Nipent®, Nitrogen Mustard, Novaldex®, Novantrone®, Nplate, Octreotide, Octreotide acetate, Ofatumumab, Oncospar®, Oncovin®, Ontak®, Onxal™, Oprelvekin, Orapred®, Orasone®, Oxaliplatin, Paclitaxel, Paclitaxel Protein-bound, Pamidronate, Panitumumab, Panretin®, Paraplatin®, Pazopanib, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolia®, Prolifeprospan 20 with Carmustine Implant, Provenge®, Purinethol®, Raloxifene, Revlimid®, Rheumatrex®, Rituxan®, Rituximab, Roferon-A® (Interferon Alfa-2a), Romiplostim, Rubex®, Rubidomycin hydrochloride, Sandostatin®, Sandostatin LAR®, Sargramostim, Sipuleucel-T, Soliris®, Solu-Cortef®, Solu-Medrol®, Sorafenib, SPRYCEL™, STI-571, Streptozocin, SU11248, Sunitinib, Sutent®, Tamoxifen, Tarceva®, Targretin®, Tasigna®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, Valrubicin, Valstar, vandetanib, VCR, Vectibix™, Velban®, Velcade®, Vemurafenib, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, Votrient, VP-16, Vumon®, Xalkori capsules, Xeloda®, Xgeva®, Yervoy®, Zanosar®, Zelboraf, Zevalin™, Zinecard®, Zoladex®, Zoledronic acid, Zolinza, Zometa®, and Zytiga®.

In one embodiment of any method or composition described herein, the at least one additional cancer therapy is selected from the group consisting of radiation therapy, chemotherapy, immunotherapy and gene therapy.

In one embodiment of any method described herein, the method further comprises administering a drug that treats at least one symptom of cancer or cancer therapy. For example, for low blood count or anemia resulting from the chemo- or radiation therapy, erythropoietin can be administered to promote de novo the production of blood cells.

In one embodiment of any method described, each compound is administered singly, i.e. each compound is administered independently of the others. In another embodiment of any method described, the compounds are administered singly and simultaneously. In another embodiment, the compounds are administered together, e.g., in a cocktail or a pharmaceutical composition.

In one embodiment of any composition described, the composition is formulated for administration by a route selected from the group consisting of: intravenous, intramuscular, subcutaneous, intradermal, topical, intraperitoneal, intrathecal, intrapleural, intrauterine, rectal, vaginal, intrasynovial, intraocular/periocular, intraorgan, intratumor, and parenteral administration.

In one embodiment of any method described herein, the method further comprises selecting a subject who has cancer or has been diagnosed with cancer. The subject can be screened for cancer with a combination with diagnostics such as, for example, additional biomarkers, mammography, manual examination, MRI, or tissue biopsy and histopathological examination. A skilled oncologist or physician will be able to differentially diagnose cancer using medical diagnostic methods known within the art.

In one embodiment of any method described herein, the method further comprises selecting a subject having mTOR deregulation or hyperactivity. In one embodiment, the mTOR deregulation results in mTOR hyperactivity. In one embodiment, the cancer involving mTOR deregulation or hyperactivity is LAM. In another embodiment, the cancer in LAM.

In one embodiment, the cancer in the subject involves mTOR deregulation or hyperactivity. In one embodiment, the mTOR deregulation results in mTOR hyperactivity. In one embodiment, the cancer involving mTOR deregulation or hyperactivity is LAM. In another embodiment, the cancer is LAM.

In one embodiment of any method described herein, the method further comprises selecting a subject who has TSC or has been diagnosed with a mutation at the TSC loci. The subject can be genetically screened for TSC. A skilled physician will be able to differentially diagnosis TSC using medical diagnostic methods known within the art.

In one embodiment of any method described herein, the subject is a mammal. In another embodiment, the subject is a primate mammal. In one embodiment of any method described, the subject is human.

### Formulation and Application

In one embodiment, the compounds or combination of compounds are delivered with a pharmaceutically acceptable carrier.

In one embodiment, the term “pharmaceutically acceptable” means approved by a regulatory agency of the Federal or a state government or listed in the U.S. Pharmacopeia or other generally recognized pharmacopeia for use in animals, and more particularly in humans. Specifically, it refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration. Additional carrier agents, such as liposomes, can be added to the pharmaceutically acceptable carrier.

Therapeutic compositions contain a physiologically tolerable carrier together with at least a compound or combination of compounds as described herein, dissolved or dispersed therein as an active ingredient. In one embodiment, the therapeutic composition is not immunogenic when administered to a mammal or human patient for therapeutic purposes. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Compositions can be prepared as injectable either as liquid solutions or suspensions, however, solid forms suitable for solution, or suspensions; in liquid prior to use can also be prepared. The preparation can also be emulsified or presented as a liposome composition. The compounds or combination of compounds can also be conjugated with lipids, e.g., amphipathic lipids, for stability and delivery purposes. The conjugation bonds are reversible and are broken or dissolved when the compounds or combination of compounds are delivered to target destination. Alternatively, the compounds or combination of compounds described herein can be prepared as a solid or semi-solid or emulsion in suppository, e.g., as microspheres. The microspheres can be inserted as a solid into or targeted to a solid tumor. The compounds or combination of compounds described herein can be mixed with excipients which are pharmaceutically acceptable and compatible with the active ingredient and in amounts suitable for use in the therapeutic methods described herein. Specifically contemplated pharmaceutical compositions are compounds or combination of compounds in a preparation for delivery as described herein above, or in references cited and incorporated herein in that section. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition comprising the compounds or combination of compounds described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of compounds or combination of compounds or composition used in the methods described herein that will be effective in the treatment of a particular disorder or condition will depend on the nature of the disorder or condition, and can be determined by standard clinical techniques.

The therapeutic compositions or pharmaceutical compositions described herein can be formulated for passage through the blood-brain barrier or direct contact with the endothelium. In some embodiments, the compositions can be formulated for systemic delivery. In some embodiments, the compositions can be formulated for delivery to specific organs, for example but not limited to the liver, spleen, the bone marrow, and the skin. The compositions or pharmaceutical compositions can also be formulated for aerosol application by inhalation into the lung. Alternatively, the therapeutic compositions or pharmaceutical compositions can also be formulated for a transdermal delivery, e. g. via a skin patch. Therapeutic compositions or pharmaceutical compositions can be enteric coated and formulated for oral delivery. Alternatively, the compositions or pharmaceutical compositions can be encapsulated in liposomes or nanoparticles and formulated for slow sustained delivery in vivo. Alternatively, the therapeutic compositions or pharmaceutical compositions is be formulated for targeted delivery, eg., encapsulated in liposomes or nanoparticles that are designed and feature targeting moiety to on the liposomes or nanoparticles.

The therapeutic compositions or pharmaceutical compositions described herein can be administered by any known route. By way of example, the therapeutic compositions or pharmaceutical compositions described herein can be administered by a mucosal, pulmonary, topical, or other localized or systemic route (e.g., enteral and parenteral). The compositions may be administered by any convenient route, for example by infusion or bolus injection, by absorption through epithelial or mucocutaneous linings (e.g., oral mucosa, rectal and intestinal mucosa, etc.) and may be administered together with other active agents.

Routes of administration include, but are not limited to aerosol, direct injection, intradermal, transdermal (e.g., in slow release polymers), intravitreal, intramuscular, intraperitoneal, intravenous, subcutaneous, epidural, topical, oral, transmucosal, buccal, rectal, vaginal, transdermal, intranasal and parenteral routes.

“Parenteral” refers to a route of administration that is generally associated with injection, including but not limited to intraorbital/periocular, infusion, intraarterial, intracapsular, intraocular, intracardiac, intraorgan, intradermal, intrahepatic, intrarogan, intramuscular, intraperitoneal, intrapulmonary, intratumor, intraspinal, intrasternal, intrathecal, intrauterine, intravenous, subarachnoid, subcapsular, intrasynovial, subcutaneous, transmucosal, or transtracheal. Any other therapeutically efficacious route of administration can be used, for example, infusion or bolus injection, absorption through epithelial or mucocutaneous linings. In various embodiments, administration can be inhaled in to the lung via aerosol administration, e.g. with nebulization. Administration also can be systemic or local, for example, intratumoral delivery is also included.

In some embodiments, the one or more compounds used for treatment or the therapeutic compositions or pharmaceutical compositions described herein are administering via a nebulizer. For example, the agent can be formulated as a powder for delivery via a nebulizer.

For example, the one or more compounds used for treatment or the therapeutic compositions or pharmaceutical compositions described herein are formulated for delivery by nebulizer. Such formulations are known in the art. For examples, nebulizer formulations are described in O'Riordan TG. et al., 2002, Respir Care. 47:1305-12; U.S. Patent publication US20070207091; and U.S. Pat. No. 7,405,207; the contents of which are incorporated by reference in their entirety. Other dary inhalation formulation are known in the art, for example, in U.S. Pat. Nos. 5,983,956, 6,027,714, and 8,071,127, and U.S. Patent publication US2003/0148925 and US2011/0236492, the contents of which are incorporated by reference in their entirety.

The precise dose and formulation to be employed depends upon the potency of the compounds or combination of compounds described herein, and depends on the amounts large enough to produce the desired effect, e.g., a reduction in size and/or growth of the tumors in the subject. The dosage should not be so large as to cause unacceptable adverse side effects. Generally, the dosage will vary with the compounds or combination of compounds, and with the age, condition, and size of the tumors in the subject are also considered. Dosage and formulation of the compounds or combination of compounds will also depend on the route of administration, and the mass and number of tumors in the subject, and should be decided according to the judgment of the practitioner and each subject's circumstances. Effective doses can be extrapolated from dose-response curves derived from in vitro or animal model test systems.

The dosage can be determined by one of skill in the art and can also be adjusted by the individual physician in the event of any complication. Typically, the dosage ranges from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 g/kg body weight to 30 g/kg body weight. Alternatively, the dose range will be titrated to maintain serum levels between 5 g/mL and 30 g/mL.

Administration of the doses recited above can be repeated for a limited period of time. In some embodiments, the doses are given once a day, or multiple times a day, for example but not limited to three times a day. In one embodiment, the doses recited above are administered daily for several weeks or months. The duration of treatment depends upon the subject's clinical progress and responsiveness to therapy, e.g., shrinkage of tumor sizes. Continuous, relatively low maintenance doses are contemplated after an initial higher therapeutic dose. As but one example, the compounds or combination of compounds and a pharmaceutically acceptable carrier can be formulated for direct application by injection into the tumor in the subject.

Efficacy testing can be performed during the course of treatment using the methods described herein, e.g., ultrasound, MRI and CT to monitor the shrinkage in size of the tumors in the treated subject. A decrease in size of the tumors during and after treatment indicates that the treatment is effective in reducing tumor size. Measurements of the degree of severity of a number of symptoms associated with cancerous tumors are also noted prior to the start of a treatment and then at later specific time period(s) after the start of the treatment. A skilled physician will be able to ascertain the tumor sizes and related symptoms by known methods in the art and those described herein.

This disclosure is further illustrated by the following example which should not be construed as limiting. The contents of all references cited throughout this application, as well as the figures and table are incorporated herein by reference.

Those skilled in the art will recognize, or be able to ascertain using not more than routine experimentation, many equivalents to the specific embodiments of the disclosure described herein. Such equivalents are intended to be encompassed by the following claims.

Some embodiments of the technology described herein can be defined according to any of the following numbered paragraphs:

1. A method for treating cancer in a subject, the method comprising:

a. determining whether cancer cells of the subject involves mTOR deregulation or hyperactivity; and, if so,

b. administering to the subject a therapeutically effective amount of a compound selected from the group consisting of nateglinide, Z-L-Phe chloromethyl ketone, clemastine fumarate, supercinnamaldehyde, practolol, fluvastatin Na, sulindac, BIO, amorolfine, spectinomucin, sibutramine HCl, nelfinavir mesylate, moroxydine HCl, nicotine ditartrate, trequinsin, megluime, tizanidine HCl, CGP-74514A hydrochloride, tioconazole, TOVOK™ (afatinib), and kasugamycin.

2. A method for treating cancer in a subject comprising administering to a subject in need thereof a therapeutically effective amount of a compound selected from the group consisting of nateglinide, Z-L-Phe chloromethyl ketone, clemastine fumarate, supercinnamaldehyde, practolol, fluvastatin Na, sulindac, BIO, amorolfine, spectinomucin, sibutramine HCl, nelfinavir mesylate, moroxydine HCl, nicotine ditartrate, trequinsin, megluime, tizanidine HCl, CGP-74514A hydrochloride, tioconazole, TOVOK™ (afatinib), and kasugamycin, wherein the cancer involves mTOR deregulation or hyperactivity.
3. The method of paragraph 1 or 2, wherein the mTOR hyperactivity is at least 10% higher compared to a control mTOR activity level.
4. The method of paragraph 3, wherein the control is an mTOR activity level in a population of normal non-cancer cells of the subject or an average mTOR activity level in a population of healthy subjects.
5. The method of paragraph 1 or 2, wherein the subject is further treated with at least one additional therapy.
6. The method of paragraph 5, wherein the at least one additional therapy is a cancer therapy.
7. The method of paragraph 6, wherein the at least one additional cancer therapy is selected from the group consisting of radiation therapy, chemotherapy, immunotherapy and gene therapy.
8. A method for treating tuberous sclerosis complex (TSC) in a subject comprising administering to a subject in need thereof a therapeutically effective amount of a compound selected from the group consisting of nateglinide, Z-L-Phe chloromethyl ketone, clemastine fumarate, supercinnamaldehyde, practolol, fluvastatin Na, sulindac, BIO, amorolfine, spectinomucin, sibutramine HCl, nelfinavir mesylate, moroxydine HCl, nicotine ditartrate, trequinsin, megluime, tizanidine HCl, CGP-74514A hydrochloride, tioconazole, TOVOK™ (afatinib), and kasugamycin.
9. The method of paragraph 1, 2, or 8, wherein the subject is human.
10. A method for treating cancer in a subject comprising administering to subject in need thereof a therapeutically effective amount of a compound selected from the group consisting of nateglinide, Z-L-Phe chloromethyl ketone, clemastine fumarate, supercinnamaldehyde, practolol, fluvastatin Na, sulindac, BIO, amorolfine, spectinomucin, sibutramine HCl, nelfinavir mesylate, moroxydine HCl, nicotine ditartrate, trequinsin, megluime, tizanidine HCl, CGP-74514A hydrochloride, tioconazole, TOVOK™ (afatinib), and kasugamycin.
11. The method of paragraph 10, wherein the cancer cells of the subject involves mTOR deregulation or hyperactivity.
12. The method of paragraph 11, wherein the cancer is lymphangioleiomyomatosis (LAM).
13. A method for inhibiting cell growth, the method comprising contacting a cell with an effective amount of a compound selected from the group consisting of nateglinide, Z-L-Phe chloromethyl ketone, clemastine fumarate, supercinnamaldehyde, practolol, fluvastatin Na, sulindac, BIO, amorolfine, spectinomucin, sibutramine HCl, nelfinavir mesylate, moroxydine HCl, nicotine ditartrate, trequinsin, megluime, tizanidine HCl, CGP-74514A hydrochloride, tioconazole, TOVOK™ (afatinib), and kasugamycin; wherein the cell has a detectable level mTOR deregulation or hyperactivity.
14. The method of paragraph 13, wherein the cell is associated with a disease selected from the group consisting of: cancer; lymphangioleiomyomatosis (LAM); angiomyolipomata (AML); Cowden's disease; Proteus syndrome; Lhermitte-Duclose disease; Peutz-Jeghers syndrome (PJS); familial hypertrophic cardiomyopathy (HCM); prostate cancer; breast cancer; lung cancer; bladder cancer; melanoma; renal cell carcinoma; ovarian cancer; endometrial cancer; thyroid cancer; glioblastoma; chronic myeloid leukemia (CML); and tuberous sclerosis complex (TSC).
15. The method of paragraph 13, wherein the method further comprises determining whether the cell has a detectable level of mTOR deregulation or hyperactivity.
16. The method of paragraph 15, wherein the mTOR hyperactivity is at least 10% higher compared to a control mTOR activity level.
17. The method of paragraph 16, wherein the control is an mTOR activity level in a population of normal cells of the subject or an average mTOR activity level in the cells of a population of healthy subjects.
18. The method of paragraph 13, wherein the cell is comprised by a subject and is contacted with the at least one compound by administering a therapeutically effective amount of the compound to the subject.
19. The method of paragraph 1, 2, 8, 10, or 13, wherein the therapeutically effective amount of the compound is administered by a route selected from the group consisting of: aerosol, direct injection, local, systemic, intradermal, direct inhalation, intravitreal, intramuscular, intraperitoneal, intravenous, intrathecal, intrapleural, intrauterine, subcutaneous, epidural, topical, oral, transmucosal, buccal, rectal, vaginal, transdermal, intranasal, intrasynovial, intraocular/periocular, intraorgan, intratumor, and parenteral administration.
20. The method of any of paragraphs 1, 2, 8, 10, or 13, wherein the therapeutically effective amount of nateglinide, Z-L-Phe chloromethyl ketone, clemastine fumarate, supercinnamaldehyde, practolol, fluvastatin Na, sulindac, BIO, amorolfine, spectinomucin, sibutramine HCl, nelfinavir mesylate, moroxydine HCl, nicotine ditartrate, trequinsin, megluime, tizanidine HCl, CGP-74514A hydrochloride, tioconazole, TOVOK™ (afatinib), or kasugamycin is administered in conjunction with at least one additional therapy to achieve a combination therapy.
21. The use of one or more compounds selected from the group consisting of nateglinide, Z-L-Phe chloromethyl ketone, clemastine fumarate, supercinnamaldehyde, practolol, fluvastatin Na, sulindac, BIO, amorolfine, spectinomucin, sibutramine HCl, nelfinavir mesylate, moroxydine HCl, nicotine ditartrate, trequinsin, megluime, tizanidine HCl, CGP-74514A hydrochloride, tioconazole, afatinib, and kasugamycin for the preparation of a medicament for the treatment of cancer.
22. The use of one or more compounds selected from the group consisting of nateglinide, Z-L-Phe chloromethyl ketone, clemastine fumarate, supercinnamaldehyde, practolol, fluvastatin Na, sulindac, BIO, amorolfine, spectinomucin, sibutramine HCl, nelfinavir mesylate, moroxydine HCl, nicotine ditartrate, trequinsin, megluime, tizanidine HCl, CGP-74514A hydrochloride, tioconazole, afatinib, and kasugamycin for the preparation of a medicament for the treatment of tuberous sclerosis complex.

### Example

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

Methods

The screen is being performed in 621-101 cells (which are derived from an angiomyolipoma (AML) of a patient with LAM and have a bi-allelic TSC2 inactivating mutation). The 621-101 cells are plated at 1250 cells/well in 384-well plates and grown overnight. Compound libraries are then added at the ICCB and allowed to incubate for 48-72 h. ATP levels (CELLTITER GLO assay, Promega) are used as an indicator of cell viability. Treatment with staurosporine (1-2 uM) represents a positive control for cell death.

Results:

The inventors screened the Known Bioactives Collection at the ICCB which consists of approximately 7,000 compounds. The screen identified and confirmed 20 compounds that are more efficacious alone than in combination with rapamycin treatment (can be utilized as single agents).

The references cited herein and throughout the specification are incorporated herein by reference.

Summary: The compounds selected from the group consisting of nateglinide, Z-L-Phe chloromethyl ketone, clemastine fumarate, supercinnamaldehyde, practolol, fluvastatin Na, sulindac, BIO, amorolfine, spectinomucin, sibutramine HCl, nelfinavir mesylate, moroxydine HCl, nicotine ditartrate, trequinsin, megluime, tizanidine HCl, CGP-74514A hydrochloride, tioconazole, TOVOK™ (afatinib), or kasugamycin are only effective when the mTOR pathway is active. Pretreatment of cells with rapamycin protects the cells against these compounds. These data indicate that these compounds will exhibit a wide therapeutic index due to the selectively high mTOR activity in TSC, LAM and certain cancers, as compared to normal tissues.

1. A method for treating cancer in a subject, the method comprising:
a) determining whether cancer cells of the subject involves mTOR deregulation or hyperactivity; and, if so,
a) administering to the subject a therapeutically effective amount of one or more compounds selected from the group consisting of nateglinide, Z-L-Phe chloromethyl ketone, clemastine fumarate, supercinnamaldehyde, practolol, fluvastatin Na, sulindac, BIO, amorolfine, spectinomucin, sibutramine HCl, nelfinavir mesylate, moroxydine HCl, nicotine ditartrate, trequinsin, megluime, tizanidine HCl, CGP-74514A hydrochloride, tioconazole, afatinib, and kasugamycin.

2. (canceled)

3. The method of claim 1, wherein the mTOR hyperactivity is at least 10% higher compared to a control mTOR activity level.

4. The method of claim 3, wherein the control is an mTOR activity level in a population of normal non-cancer cells of the subject or an average mTOR activity level in a population of healthy subjects.

5. The method of claim 1, wherein the subject is further treated with at least one additional therapy.

6. The method of claim 5, wherein the at least one additional therapy is a cancer therapy.

7. The method of claim 6, wherein the at least one additional cancer therapy is selected from the group consisting of radiation therapy, chemotherapy, immunotherapy and gene therapy.

8. A method for treating tuberous sclerosis complex (TSC) in a subject comprising administering to a subject in need thereof a therapeutically effective amount of one or more compounds selected from the group consisting of nateglinide, Z-L-Phe chloromethyl ketone, clemastine fumarate, supercinnamaldehyde, practolol, fluvastatin Na, sulindac, BIO, amorolfine, spectinomucin, sibutramine HCl, nelfinavir mesylate, moroxydine HCl, nicotine ditartrate, trequinsin, megluime, tizanidine HCl, CGP-74514A hydrochloride, tioconazole, afatinib, and kasugamycin.

9. The method of claim 8, wherein the subject is human.

10. (canceled)

11. (canceled)

12. The method of claim 1, wherein the cancer is lymphangioleiomyomatosis (LAM).

13. A method for inhibiting cell growth, the method comprising contacting a cell with an effective amount of one or more compounds selected from the group consisting of nateglinide, Z-L-Phe chloromethyl ketone, clemastine fumarate, supercinnamaldehyde, practolol, fluvastatin Na, sulindac, BIO, amorolfine, spectinomucin, sibutramine HCl, nelfinavir mesylate, moroxydine HCl, nicotine ditartrate, trequinsin, megluime, tizanidine HCl, CGP-74514A hydrochloride, tioconazole, afatinib, and kasugamycin; wherein the cell has a detectable level mTOR deregulation or hyperactivity.

14. The method of claim 13, wherein the cell is associated with a disease selected from the group consisting of: cancer; lymphangioleiomyomatosis (LAM); angiomyolipomata (AML); Cowden's disease; Proteus syndrome; Lhermitte-Duclose disease; Peutz-Jeghers syndrome (PJS); familial hypertrophic cardiomyopathy (HCM); prostate cancer; breast cancer; lung cancer; bladder cancer; melanoma; renal cell carcinoma; ovarian cancer; endometrial cancer; thyroid cancer; glioblastoma; chronic myeloid leukemia (CML); and tuberous sclerosis complex (TSC).

15. The method of claim 13, wherein the method further comprises determining whether the cell has a detectable level of mTOR deregulation or hyperactivity.

16. The method of claim 15, wherein the mTOR hyperactivity is at least 10% higher compared to a control mTOR activity level.

17. The method of claim 16, wherein the control is an mTOR activity level in a population of normal cells of the subject or an average mTOR activity level in the cells of a population of healthy subjects.

18. The method of claim 13, wherein the cell is comprised by a subject and is contacted with the at least one compound by administering a therapeutically effective amount of the compound to the subject.

19. The method of claim 1, wherein the therapeutically effective amount of the compound is administered by a route selected from the group consisting of: aerosol, direct injection, local, systemic, intradermal, direct inhalation, intravitreal, intramuscular, intraperitoneal, intravenous, intrathecal, intrapleural, intrauterine, subcutaneous, epidural, topical, oral, transmucosal, buccal, rectal, vaginal, transdermal, intranasal, intrasynovial, intraocular/periocular, intraorgan, intratumor, and parenteral administration.

20. The method of claim 8, wherein the therapeutically effective amount of nateglinide, Z-L-Phe chloromethyl ketone, clemastine fumarate, supercinnamaldehyde, practolol, fluvastatin Na, sulindac, BIO, amorolfine, spectinomucin, sibutramine HCl, nelfinavir mesylate, moroxydine HCl, nicotine ditartrate, trequinsin, megluime, tizanidine HCl, CGP-74514A hydrochloride, tioconazole, afatinib, or kasugamycin is administered in conjunction with at least one additional therapy to achieve a combination therapy.

21. (canceled)

22. (canceled)
